(19) United States
(12) Patent Application Publication
(10) Pub. No.: US 2025/0314594 A1
(43) Pub. Date: Oct. 9, 2025

(54) METHOD OF DETERMINING A NUMERICAL ANALYTE RESULT VALUE

(71) Applicant: Roche Diabetes Care, Inc., Indianapolis, IN (US)

(72) Inventors: Max Berg, Mannheim (DE); Fredrik Hailer, Limburgerhof (DE); Bernhard Limburg, Soergenloch (DE)

(21) Appl. No.: 19/241,606

(22) Filed: Jun. 18, 2025

Related U.S. Application Data
(63) Continuation of application No. PCT/EP2023/086510, filed on Dec. 19, 2023.

(30) Foreign Application Priority Data
Dec. 21, 2022 (EP) 22215267.0

(51) Int. Cl. G01N 21/78

(52) U.S. Cl. CPC G01N 21/78

(57) ABSTRACT

A computer-implemented method of determining a numerical analyte result value corresponding to a concentration of an analyte in a sample of a bodily fluid of a subject applied to a reagent test region, that includes step (a) determining a numerical analyte result value based on an image of the reagent test region by using an algorithm which takes into account one or more parameters, each parameter being adapted to take more than one value, step (b) displaying the numerical analyte result value and/or a corresponding analyte value range and/or a corresponding message on a display device and step (c) automatically checking for available updated values for one or more of the parameters on a remote server before executing step (a), and, for each available updated value, updating the corresponding parameter value to be taken into account in step (a).

### CROSS REFERENCE TO RELATED APPLICATIONS

This application is a continuation of PCT/EP2023/086510 filed Dec. 19, 2023, which claims priority from EP 22 215 267.0 filed Dec. 21, 2022, the disclosures of both of which are hereby incorporated herein by reference.

### TECHNICAL FIELD

The present application refers to a computer implemented method of determining a numerical analyte result value corresponding to a concentration of an analyte in a sample of a bodily fluid of a subject applied to a reagent test region comprising steps, wherein a numerical analyte result value is determined based on an image of the reagent test region by using an algorithm which takes into account one or more parameters, each parameter being adapted to take more than one value, and the numerical analyte result value and/or an analyte value range of a group of preset analyte value ranges corresponding to the numerical analyte result value and/or a message corresponding to the corresponding numerical analyte result value or the analyte value range is displayed on the display device.

### BACKGROUND

In the field of medical diagnostics, in many cases, one or more analytes have to be detected in samples of a body fluid, such as blood, interstitial fluid, urine, saliva or other types of body fluids. Examples of analytes to be detected are glucose, triglycerides, lactate, cholesterol or other types of analytes typically present in these body fluids. According to the concentration and/or the presence of the analyte, an appropriate treatment may be chosen, if necessary. Without narrowing the scope, the invention specifically will be described with respect to blood glucose measurements. It shall be noted, however, that the present invention may also be used for other types of analytical measurements using a test element with a reagent test region. Generally, devices and methods known to the skilled person make use of test elements comprising one or more test chemistries, which, in presence of the analyte to be detected, are capable of performing one or more detectable detection reactions, such as optically detectable detection reactions. With regard to these test chemistries, reference may be made e.g. to J. Hoenes et al.: The Technology Behind Glucose Meters: Test Strips, Diabetes Technology & Therapeutics, Volume 10, Supplement 1, 2008, S-10 to S-26. Other types of test chemistry are possible and may be used for performing the present invention.

Typically, one or more optically detectable changes in the test chemistry are monitored, for example, a change of color of the reagent test region. In analytical measurements based on such a color formation reaction, one technical challenge resides in the evaluation of the occurring color change. Besides using dedicated analytical devices, such as handheld blood glucose meters, the use of generally available electronic devices such as smart phones and portable computers has become more and more popular over the recent years. Such a method for detecting an analyte concentration using images of the reagent test region generated by a mobile device is known from EP 3 591 385 A1. Another method for analyte concentration detection using a mobile device is disclosed in EP 3 978 909 A1, wherein in the field of view of the mobile device's camera the reagent test region is combined with a color reference card comprising a plurality of different color reference fields having known reference color values and/or gray field and the known reference values are accounted for when determining the concentration of the analyte in the applied sample. The determination of the concentration of the analyte may be performed by the mobile device, wherein necessary parameters such as the reference values may be stored in an internal data storage of the mobile device and/or received from a remote server in advance of the determination process.

Alternatively, as disclosed in CN 113 569 678 A for an automatic biochemical index detection method for pet urine using a urine test paper only the image acquisition and result presentation is performed by the mobile device, while image processing and result value determination is done by a remote server.

For the accuracy of the image processing and result value determination, it is crucial that all parameters used are up-to-date.

It is therefore desirable to provide a method and device, which address the above-mentioned technical challenge of analytical measurements using mobile devices. Specifically, methods, computer programs, and devices shall be proposed which are widely applicable to available mobile devices and which are suited to improve reliability and safety while allowing convenient handling for the user and being resource-efficient.

### BRIEF SUMMARY

This problem is addressed by methods, computer programs, and devices with the features described herein. Advantageous embodiments, which might be realized in an isolated fashion or in any arbitrary combination, are also described. One aspect of the disclosure is to automatically check for available updated values for one or more of parameters, before using one or more of the parameters when determining a numerical analyte result value via an algorithm.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once.

Further, as used in the following, the terms “preferably”, “more preferably”, “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

In one aspect, a computer implemented method of determining a numerical analyte result value corresponding to a concentration of an analyte in a sample of a bodily fluid of a subject applied to a reagent test region includes step (a) of determining a numerical analyte result value based on an image of the reagent test region by using an algorithm which takes into account one or more parameters, each parameter being adapted to take more than one value, step (b) of displaying the numerical analyte result value and/or an analyte value range of a group of preset analyte value ranges corresponding to the numerical analyte result value and/or a message corresponding to the corresponding numerical analyte result value or the analyte value range, and step (c) of automatically checking for available updated values for one or more of the parameters on a remote server before executing step (a), and for each available updated value updating the corresponding parameter value to be taken into account in step (a).

In another aspect, a method of determining a numerical analyte result value corresponding to a concentration of an analyte in a sample of a bodily fluid of a subject applied to a reagent test region and determined using a mobile device having a processor, a communication interface and a display device, includes step (a) of determining, by the processor, a numerical analyte result value based on an image of the reagent test region by using an algorithm which takes into account one or more parameters, each parameter being adapted to take more than one value, step (b) of displaying, by the display device, the numerical analyte result value and/or an analyte value range of a group of preset analyte value ranges corresponding to the numerical analyte result value and/or a message corresponding to the corresponding numerical analyte result value or the analyte value range, and step (c) of automatically checking, by the processor, via the communication interface for available updated values for one or more of the parameters on a remote server before executing step (a), and for each available updated value updating, by the processor, the corresponding parameter value to be taken into account in step (a).

In another aspect a non-transitory computer-readable storage medium includes instructions that, when processed by a computing device comprising a processor and a display device, configure the computing device to perform one or both of the aforementioned methods.

In another aspect, a computing device includes a processor, a display device and a communication interface. The computing device also includes a memory storing instructions that, when executed by the processor, configure the computing device to determine, by the processor, a numerical analyte result value based on an image of the reagent test region by using an algorithm which takes into account one or more parameters, each parameter being adapted to take more than one value, display, by the display device, the numerical analyte result value and/or an analyte value range of a group of preset analyte value ranges corresponding to the numerical analyte result value and/or a message corresponding to the corresponding numerical analyte result value or the analyte value range on the display device, and before executing step (a) automatically check, by the processor, for available updated values for one or more of the parameters on a remote server, and for each available updated value updating the corresponding parameter value to be taken into account in step (a).

“Computing device” here refers to any electronic machine or data processing apparatus that can be programmed or carry out a program such as a computer implemented method. In particular, a computer device may be a personal computer or a mobile device, such as a mobile phone or tablet, or the like comprising one or more data processing devices such as one or more data processor.

“Analyte” refers to a substance or chemical constituent that is of interest in an analytical procedure and is also referred to as component or chemical species. As an example, one or more analytes may be determined which take part in metabolism, such as blood glucose. Additionally, or alternatively, other types of analytes or parameters may be determined, e.g. a pH value. The analyte is comprised in at least one sample of a bodily fluid of a subject, i.e., a patient, and causes a test chemical, also referred to as a test reagent, to undergo a color formation reaction. To cause the reaction, the bodily fluid is applied to a reagent test region, which, without limitation, may be part of a test element, such as a test strip or the like and is capable of a color formation reaction when in contact with the respective analyte.

“Sample” refers to a limited quantity of something, which is intended to be similar to and represent a larger amount of that thing(s). “Bodily fluid” refers to any liquid within the body, such as blood, interstitial fluid, urine, saliva or the like. “Color formation” refers to a color of the reagent test region resulting from a color formation reaction, i.e., a chemical, biological or physical reaction during which a color, specifically a reflectance, of at least one element involved in the reaction, changes depending on the concentration of the analyte involved in the reaction. The term “color” as used herein is a broad term and may refer to any form of the light reflected of a reagent test region comprising the color. In particular, the term “color” also refers to black, white, or grey, as well as to red or green or blue, etc. By the color formation, the concentration of the analyte in the bodily fluid may be determined by evaluating the color formation, i.e., the color change due to the reaction.

The concentration of the analyte, in form of a numerical analyte result value, is determined based on an image of the reagent test region, in particular of the reagent test region after the sample has been applied, and the color formation reaction has occurred. In one embodiment, the image may be recorded by a camera, e.g., by a camera comprised by the mobile device. “Mobile device” refers to a mobile electronics device, such as a consumer-electronics mobile device, specifically multipurpose mobile devices that are not dedicated to analytical measurements. A mobile device may refer to a portable device with at least one processor, at least one display device, and optionally a camera. The mobile device may specifically refer to a mobile communication device such as a cell phone or smartphone. Additionally, or alternatively, the mobile device may also refer to a tablet computer or another type of portable computing device having a display device and, optionally, a camera. “Processor” refers to any device or combination of machines capable of processing data, i.e., producing for a set of inputs a defined set of outputs. For example, a processor may be a central processing unit (CPU), e.g. a microprocessor, and/or a multi-core processor.

“Camera” refers to a device having at least one imaging element configured for recording or capturing spatially resolved one-dimensional, two-dimensional, or even three-dimensional optical data or information. As an example, the camera may comprise at least one camera chip, such as at least one CCD chip and/or at least one CMOS chip configured for recording images. “Image” refers to a set of spatially resolved optical data. Specifically, the term may relate to data recorded by using a camera, such as a plurality of electronic readings from the imaging device, such as the pixels of the camera chip. Further, the term may refer, without limitation, to a color digital image made of pixels, each pixel comprising color information, such as color values, for at least three different colors, i.e., at least three different wavelengths of light. A larger number of color values is also feasible, such as four color values for each pixel, for example R, G, G, B.

Color cameras as well as images, in particular color digital images, are generally known to the skilled person. Thus, as an example, the camera chip may consist of a plurality of three or more different color sensors each, such as color recording pixels like one pixel for red (R), one pixel for green (G) and one pixel for blue (B). For each of the pixels, such as for R, G, B, values may be recorded, such as digital values in the range of 0 to 255, depending on the intensity of the respective color, the plurality of recorded pixels resulting in an image. Instead of using color triples such as R, G, B, as an example, quadruples may be used, such as R, G, G, B. The color sensitivities of the pixels may be generated by color filters or by appropriate intrinsic sensitivities of the sensor elements used in the camera pixels. The skilled person generally knows these techniques.

The determined numerical analyte result value is then communicated to a user by displaying the numerical analyte result value itself, and/or an analyte value range and/or a message corresponding to the determined numerical analyte result value. A corresponding or associated “message” refers to any displayable sign capable of informing the user of the status of the analyte concentration, i.e., of the determined numerical analyte result value. The message may comprise one or more of a range name, corresponding values like limit values of a range, a corresponding recommendation, a symbol, and the like. The communication may be realized via a display device, wherein “display device” refers to any output device for communication of information to a user in visual or tactile form, such as, without limitation, LCD displays, LED displays, or tactile electronics displays and which may serve to communicate a result, such as the numerical analyte result value.

The numerical analyte result value corresponding to the concentration is determined based on the image using an algorithm, wherein the algorithm typically takes into account a number of parameters. “Algorithm” generally refers to a finite sequence of rigorous instructions, typically used to solve a class of specific problems or to perform a computation. Here the algorithm is a sequence of instructions to process the image and to calculate the numerical analyte result value on the bases of the image processing. “Parameter” here refers to a value used by the algorithm that may take more than one value, i.e., that may change over time. The change may occur on a regular basis, e.g., adaptions to a specific production lot of a component used, or non-regularly, e.g., be pursued in the context of product care to improve the method. For example, one or more of the parameters may each represent a characteristic of one of the used components or the like, such as a correlation between a color of the reagent test region and a concentration or concentration range of an analyte, or a value for a safety margin to reduce an area of the reagent test region in the image taken into account for the determination of the analyte concentration, or a reference color value of a color reference device if used, or a failsafe threshold value, or the like. For example, if one of the components changes, it may be beneficial or necessary to update the corresponding parameter, i.e., the parameter needs to take a new value differing from the former value. It may also be advantageous to change parameters on the basis of knowledge from an analysis of historic numerical analyte result values determined by the method and/or other user data. The updating generally enables a constant improvement of the method.

The parameters are stored in a data storage, wherein “data storage” or memory refers to any type of digital data storage technology such as a random access memory, a hard disk mass storage device or a removable medium via a corresponding drive or the like or any combination thereof. In an embodiment, the computing device, e.g. the mobile device, comprises the data storage.

An aspect of the invention is to automatically check if updated values for one or more of the parameters are available on a remote server, i.e., if a remote server can be reached and if any of the values provided by the remote server differ from the current parameter values accounted for by the algorithm. If the remote server can't be reached, i.e., no communication may be established and/or the remote server does not answer a request sent under method step (c), no updated values for any of the parameters are available and the current parameter values are used in step (a).

“Remote server” refers to a piece of computer hardware or software that provides functionality for other programs or devices often-called clients. For example, the remote server may be or may comprise at least one cloud-based server, also referred to as a cloud-based server device. Other embodiments are feasible. The remote server may directly or indirectly communicate with a client via a wireless network, e.g., the internet or a local network, and/or via a wired connection. Furthermore, the remote server may provide one or more functionalities such as sharing data and/or performing computation for a client. In an embodiment the connection to the remote server is established via a communication interface comprised by the device that carries out the method, e.g. of the mobile device, via an in-built antenna or a wireless access point. “Communication interface” or port for communication refers to any wired or wireless connection to exchange data between two or more separate components, i.e., to transmit and/or receive data. The term communication interface specifically may refer, without limitation, to an arbitrary device configured at least to receiving data, such as a wireless or wire-bound interface for receiving data. A bi-directional communication interface may be established by an arbitrary device configured to receive and transmit data, such as a transmitter, or by a receiving device and a transmission device being fully or partially separate devices.

For each available updated value the value of the corresponding parameter is updated, i.e., the former value is replaced by the available updated value. The automatic check and updating of parameters of step (c) is performed before step (a), thus before using one or more of the parameters when determining the numerical analyte result value via the algorithm.

The method ensures that all parameters used to determine the analyte concentration, i.e., the numerical analyte result value, have an up-to-date value as possible while also ensuring that the method is adapted to being performed on a local device without any network connection if necessary. If a connection to the remote server may be established the updating is resource-efficient as an update of the whole algorithm, e.g., a whole application or program, containing the parameters, is avoided. In particular, the updating is restricted to those parameters for which an update is available.

For example, the method enables to reliably update one or more parameters provided by the remote server based on determined numerical analyte result values, e.g., based on data analysis of a plurality of numerical analyte result values, the plurality of values may be chosen based on a certain time, a specific user or group of users or the like. Alternatively or additionally, one or more parameters on the remote server are updated based on information related to a color reference devices, e.g., accounting for lot specific changes of the color reference device.

The method may also include where checking for available update values includes checking on the remote server for each parameter a flag associated with the parameter, or checking on the remote server at least one status change flag, such as a single flag indicative of any status change for all parameters or for a group of parameters, or checking on the remote server a status register or flag register, such as a list of all status changes, or comparing each parameter with a corresponding parameter on the remote server. The checking may also include any combination of the aforementioned alternatives, for example and without limiting the scope, for a first group of parameters a status change flag associated with said group of parameters is checked and for a second group of parameters each parameter is compared with a corresponding parameter on the remote server. The checking may be performed by a processor, e.g., by the processor of the mobile device.

“Flag” refers to a bit field or bit fields used to control or to indicate the outcome of particular operations. For example, a hash function may be used, wherein hash function refers to any function that can be used to map data of arbitrary size to fixed-size values, the so-called hash value, hash code, digest, hash flag or simply hash that are usually used to index a fixed-size hash table. Hashing is a computationally and storage space-efficient example of a data access that may be used for checking for available updated values.

The method may also include where checking for available update values includes automatically identifying an identification code of a color reference device in the image of the reagent test region, and checking for available updates of one or more parameters linked to the identification code, therewith ensuring that all parameter linked, i.e., associated with the color reference device, are up to date. For example, the color reference device may comprise color fields with known reference color values, wherein those reference color values may, for example, slightly differ or be changes with regard to different production lots, so that an adaption of those associated or linked parameters is necessary when a new or different color reference device is used. The automatic identification may as well as the checking be performed by a processor, the same processor, or different processors, e.g., by the processor of the mobile device.

“Color reference device” refers to an arbitrary item having, disposed therein, or disposed thereon, such as on at least one surface, one or more fields having defined color levels, e.g., known reference color values. As an example, the color reference device may be a flat card comprising at least one substrate having, on at least one surface and/or disposed therein, one or more color reference fields each having a known color coordinate or value. A field may for example be a 2-dimensional structure, such as a rectangle, a square, a polygon, a circle and/or an ellipse, with a uniform color value, e.g., a uniform gray value. The color value of the field may for example be known in terms of the RGB color model or additive color model, i.e., by known values for red, green, and blue. Specifically, the color value may be one or more of predetermined, known, or determinable. For example, gray values result when a red, green, and blue color value or color channel for an image point/pixel have equal values. If using a color reference device the image forming the basis for step (a) of the method comprises, i.e., covers at least a part of the reagent test region as well as at least a part of the color reference device. Furthermore, the known reference color values are parameters used by the algorithm to increase the reliability of the determination the color of the reagent test region in said image.

“Identification code” refers to an optical marker identifying the color reference device, e.g., uniquely or as part of a group, for example a production lot, and therewith enabling to differentiate between different devices or groups of devices. The identification code may for example be a QR-code or the like. The code may or may not comprise further information.

The method may also include where at least one parameter is a reference color value of a color field of a color reference device, a failsafe threshold value, or a Region of interest margin value.

“Region of interest margin value” or ROI margin value or test field margin refers to a value for a safety margin to reduce a region of interest in the image taken into account for the determination of the analyte concentration. The region of interest is, for example, an area of the reagent test region, or an area of a color reference device if used, or the like.

The method may also include where, when in step (c) a connection to the remote server fails, step (a) is executed using the present parameter values and a message indicating the failure to establish a connection to the remote server is displayed. The message may be displayed via any display device, e.g., the display device of the mobile device.

The method may also include where the method includes the step of capturing the image for step (a).

The computing device may also include where the image of the reagent test region is recorded by a camera comprised by the computing device. Other technical features may be readily apparent to one skilled in the art from the following figures, descriptions, and claims.

The non-transitory computer-readable storage medium may also include where step (a) and (c) are performed by the processor and step (b) is performed by the display device.

The non-transitory computer-readable storage medium may also include where the image of the reagent test region is recorded by a camera comprised by the computing device. Other technical features may be readily apparent to one skilled in the art from the following figures, descriptions, and claims.

### BRIEF DESCRIPTION OF THE SEVERAL VIEWS OF THE DRAWINGS

Further optional features and embodiments will be disclosed in more detail in the subsequent description of embodiments. Therein, the respective optional features may be realized in an isolated fashion as well as in any arbitrary feasible combination, as the skilled person will realize. The scope of the invention is not restricted by the preferred embodiments. The embodiments are schematically depicted in the figures.

To easily identify the discussion of any particular element or act, the most significant digit or digits in a reference number refer to the figure number in which that element is first introduced.

FIG. 1 illustrates an aspect of the subject matter in accordance with one embodiment.

FIG. 2 illustrates an aspect of the subject matter in accordance with a further embodiment.

FIG. 3 illustrates an aspect of the subject matter in accordance with one embodiment.

### DETAILED DESCRIPTION

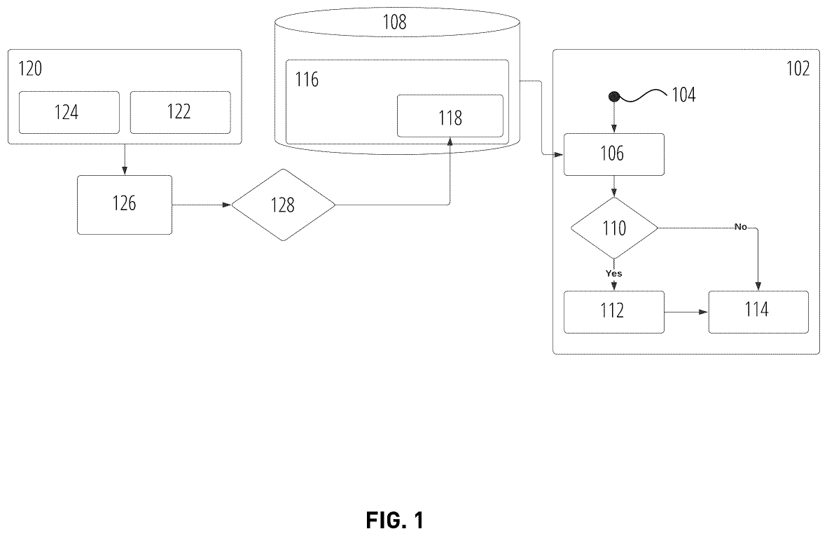

In FIG. 1 a computer-implemented method 102 is schematically depicted, the method 102 determines a numerical analyte result value corresponding to a concentration of an analyte in a sample of a bodily fluid of a subject. The determination of a numerical analyte result value corresponding to the analyte concentration is based on an image of a color formation reaction of a reagent test region to which the analyte sample has been applied. In an embodiment, the reagent test region is positioned next to a color reference device and the image is taken of both, the reagent test region and the color reference device, wherein the color reference device comprises at least one area, field, or the like with a color with a known reference color value.

The numerical analyte result value is determined by using an algorithm which takes into account one or more parameters, wherein for each parameter a value is stored and provided locally by a local data storage. The parameter, all parameters, or some of the parameters may be specific for one or more components used when executing the method 102, e.g., characteristics of a reagent of the reagent test region, or characteristics of a camera used to take the image of the reagent test region, or the like. The parameters may be grouped in different groups, e.g., in a group of regularly changing parameters and/or a group of component specific parameters, in particular a group of parameters specific to a specific production lot of the component or components. Another group may comprise parameters not changing on a regular basis, e.g., parameters of a more general nature. Said other group may comprise parameters that do not depend on any component or do not change in a relevant rate between production units of a component.

The method 102 may be triggered on a computing device, such as a mobile device, by a measurement request 104, for example by a user starting the method 102 via an input device such as a touch display of the mobile device or by a user taking with a camera of the mobile device a picture of a test element, such as a test strip, comprising a reagent test region as well as a one or two dimensional computer readable code, such as a QR code or an ArUco code or the like, comprising a trigger message, such as the measurement request 104 to start the method 102.

A method step (c) may be executed as a first step comprising establishing communication 106 with a remote server 108 and checking 110 if there are updated values for one or more of the parameters available on the remote server 108. The establishing communication 106 may comprise a uni- or bi-directional communication via a communication interface of the mobile device, such as a transmitter. The establishing communication 106 may further comprise downloading information from the remote server 108 such as the availability of updated values for parameters, all updated parameters, or all available parameters.

The checking 110 for the availability of updated parameter values may comprise comparing a group of locally stored parameters or all locally stored parameters with corresponding parameters on the remote server 108. Alternatively, the remote server 108 provides a register, list or the like indicating some or a group or all parameters that have been changed. In a further embodiment, the remote server 108 provides a list for one, more, or all parameters indicating the date a current parameter value has been stored for the parameter on the remote server.

The method step (c) further comprises updating 112 of the locally stored values of the parameters for which an updated parameter value is available. After the updating 112 or, if the checking 110 revealed no available updated parameter value on the remote server 108, after the checking 110 in a step running requested measurement 114 the numerical analyte result value is determined according by method steps (a) and (b), i.e., by determining a numerical analyte result value based on an image of a reagent test region after the application of a sample of an analyte by using the algorithm which takes into account one or more or all of the locally stored parameters and displaying the numerical analyte result value and/or an analyte value range of a group of preset analyte value ranges corresponding to the numerical analyte result value and/or a message corresponding to the corresponding numerical analyte result value or the analyte value range on a display device.

In the depicted embodiment, on the remote server 108 the parameters for the algorithm are stored in a group of current parameters 116. In the depicted embodiment, the group of current parameters 116 comprises all parameters, i.e., the current values of all parameters the method 102 is taking into account. Furthermore, the group of current parameters 116 comprises a sub-group of component-independent parameters 118. For the parameters comprised by group of current parameters 116 but not by the sub-group of component-independent parameters 118 the checking 110 comprises comparing each locally stored parameter value with the corresponding parameter value on the server. The sub-group of component-independent parameters 118 comprise parameters that are independent or at least mostly independent of any used component and therefore at least do not regularly change due to changes of components within production tolerances or the like. For the sub-group of component-independent parameters 118 a change flag indicates if any of the parameters of the sub-group of component-independent parameters 118 has been changed and the checking 110 comprises checking said change flag. The parameter values of the sub-group of component-independent parameters 118 are only compared to the locally stored parameter values if the change flag indicates that at least one parameter value of the sub-group of component-independent parameters 118 has been changed.

It is understood that in other embodiments the parameters the algorithm takes into account may as well be grouped in a single group without a sub-group or with two or more sub-groups, or in two or more separate groups, or the like on the remote server 108. Furthermore, it is understood that in other embodiments the checking 110 may comprise identical steps with regard to all parameters or may as well comprise any combination of the steps described for checking 110.

A value of one or more of the sub-group of component-independent parameters 118 is changed, for example, based on efforts in the context of maintenance and product care 120. In the depicted embodiment, the maintenance and product care 120 comprises data analysis 122, for example, analysis of all or some of the numerical analyte result values previously determined via the method 102 by one or some or all users. Furthermore, the maintenance and product care 120 comprises user feedback 124. Based on the maintenance and product care 120 and/or the user feedback 124 one or more of the parameters of the group of sub-group of component-independent parameters 118 are improved in a step of parameter improvement 126, thus based on an analysis of result data and/or feedback from users or the like a new value is determined for one or more parameter(s) of the sub-group of component-independent parameters 118. In the depicted embodiment, the parameter value is not directly and/or automatically updated in the sub-group of component-independent parameters 118 on the remote server 108 but a decision step 128 is incorporated which allows to take into account other aspects like timing or safety or authorization issues or the like. If no issues regarding safety or authorization or timing are present and a positive decision is rendered in the decision step 128 the corresponding parameter value or parameter values on the remote server 108 are updated with the improved parameter value(s) that resulted from the parameter improvement 126.

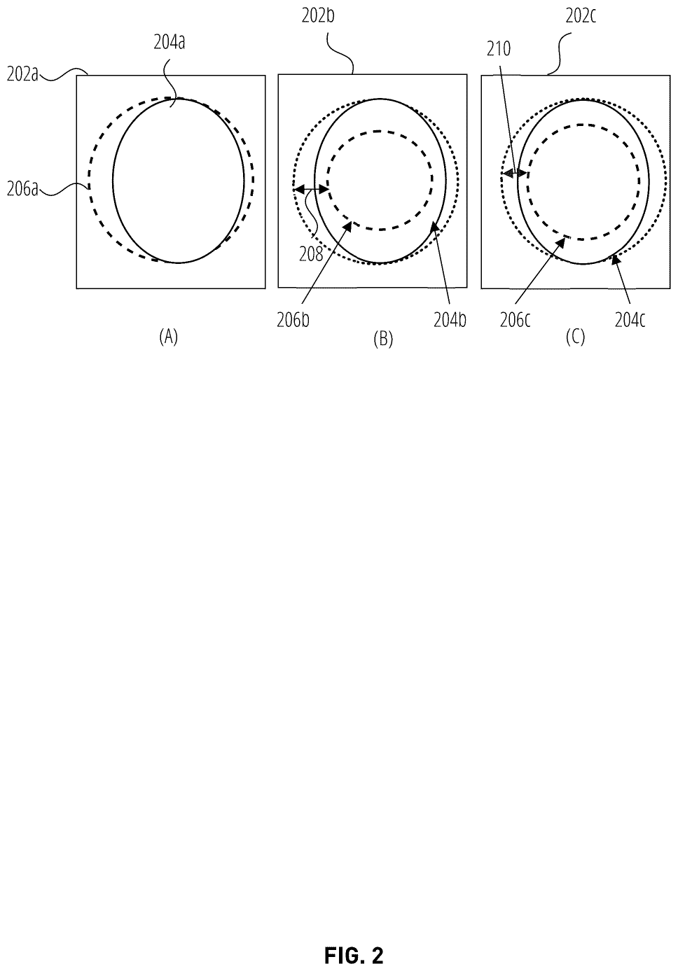

In FIG. 2 an example of a parameter of the sub-group of component-independent parameters 118 and an exemplary parameter improvement 126 are illustrated.

FIG. 2A depicts an image section 202a comprising a reagent test region 204a. In a first step, an area of the image that shall form the bases for the determination of the numerical analyte result value in step (a) of the method 102 is determined as a region of interest 206a. In the depicted embodiment, the region of interest 206a is circular and may for example be determined by image processing, such as a test field detection, e.g. using a circle detection algorithm.

As depicted in FIG. 2B in a next step the region of interest 206b is reduced to exclude all areas surrounding the reagent test region 204a to increase reliability of the method 102, so that the region of interest 206b only comprises, i.e., covers, parts of the image section 202b that show the reagent test region 204b and no surrounding areas, e.g., parts of a color reference device or the like. The region of interest 206 as determined in the first step, depicted by the dotted line in FIG. 2B, is reduced by a region of interest margin 208 having a region of interest margin value to form the reduced region of interest 206b. The region of interest margin 208 is a parameter of the sub-group of component-independent parameters 118. The region of interest margin 208, i.e., the region of interest margin value, is, for example, an absolute or relative value reducing a diameter of the region of interest 206b.

The region of interest margin 208 is a tool to reduce the image to an area within the border of the test field margin that comprises the information of the color formation and is suitable to form the bases for the determination of the numerical analyte result value. In the embodiment depicted in FIG. 2B the reduction also includes a border areas of the reagent test region 204b. This may be advantageous as border areas of the reagent test region 204b may be shadowed by a surrounding component such as a color reference device, may not be fully wetted by the analyte, or may be affected in another way. The resulting reduced region of interest 206b only covers a part of the reagent test region 204b that may form a reliable base for determining the color formation in step (a) of the method 102 and may therefore be a good choice as a starting point. Thus, the corresponding region of interest margin 208 may be used as current parameter value for the region of interest margin 208, as it reliably excludes any surrounding area as well as the border area of the reagent test region 204b.

As depicted in FIG. 2C, an analysis of a number of images taken by one or different users, e.g., during the step of data analysis 122, may, for example, reveal that a reduced region of interest margin 210, i.e., reducing the current region of interest margin 208, still reliably excludes any shadowed or otherwise affected border areas of the reagent test region 204c as well as the surrounding. Thus, a correspondingly increased region of interest 206c may be achieved without any negative effects. In this case, the region of interest margin value on the remote server may be updated to correspond to the new reduced region of interest margin 210 and the increased region of interest 206c.

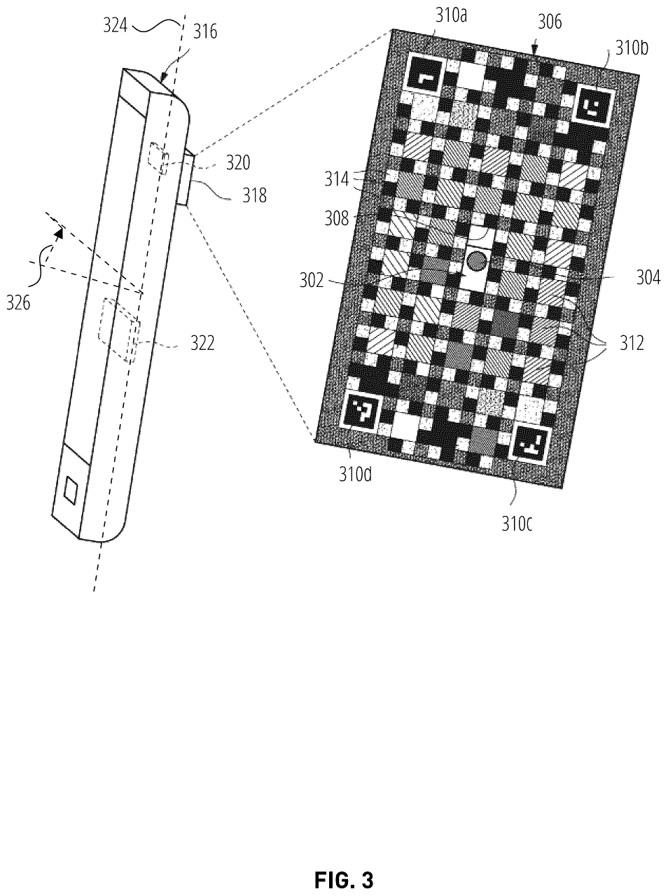

In FIG. 3 an example of a parameter of the sub-group of component-independent parameters 118 and an exemplary parameter improvement 126 are illustrated, the parameter being a maximum tilt angle.

In the depicted embodiment, a test strip 302 with a reagent test region 304 is provided surrounded by a color reference device 306. The reagent test region 304 is positioned within a window 308 of the color reference device 306. The color reference device 306 comprises position marker 310a-d, a first type of color reference field 312, e.g., reference fields having different types of green reference color value, and a second type of color reference field 314, e.g., reference fields having different types of grey reference color values. In an embodiment, the position markers 310a-d, one of the position markers 310a-d, or more of the position markers 310a-d also serve as identification code, i.e., additionally to information regarding the positioning of the color reference device 306 also comprises information to identify the color reference device 306, e.g., information regarding the production lot, type, and so forth.

To generate an image of the reagent test region 304 surrounded by the color reference device 306 a computing device 316 is provided comprising a camera 318 with a light source 320 and a processor 322. The computing device may be, for example, a mobile device such as a mobile phone or a tablet. When the user positions the camera 318, i.e., the computing device 316, to take the image the quality and/or reliability of the determination method 102 may be increased if the camera 318 is aligned in parallel to the test strip 302 and/or the color reference device 306. Therefore, in the depicted embodiment, for each image taken by the camera 318 a tilt angle 326 of the camera 318 of the computing device 316 around a longitudinal axis 324 of the computing device 316 is determined and compared to the maximum tilt angle as predefined threshold value before the image is used in step (a) of the method 102. The tilt angle 326 is measured with regard to a plane that is parallel to a plane of the test strip 302 and/or the color reference device 306. If the determined tilt angle 326 exceeds the maximum tilt angle said taken image is not used and the user is asked to take a new image.

The described safety/quality measure may result in an unpleasant number of error messages, i.e., requests to take a new image, for some users that struggle to align the camera 318 and/or not to wiggle during the recording of the image. For those users it is beneficial to analyze in the step of data analysis 122 or the like if the determined tilt angles 326 that do not comply with the threshold value for said user or a group of users or all users are typically close to the threshold, i.e., to the maximum tilt angle. In that case, an adaption of the maximum tilt angle, i.e., a slight increase of the parameter value of the maximum tilt angle, in particular for said user may significantly decrease the number of error messages received by said user, without a significant reduction of the safety and/or quality of the method 102.

LISTING OF DRAWING ELEMENTS

102 method
104 measurement request
106 establishing communication
108 remote server
110 checking
112 updating
114 running requested measurement
116 group of current parameters
118 sub-group of component-independent parameters
120 maintenance and product care
122 data analysis
124 user feedback
126 parameter improvement
128 decision step
202a image section
202b image section
202c image section
204a reagent test region
204b reagent test region
204c reagent test region
206a region of interest
206b region of interest
206c region of interest
208 region of interest margin
210 reduced region of interest margin
302 test strip
304 reagent test region
306 color reference device
308 window
310a position marker
310b position marker
310c position marker
310d position marker
312 first type of color reference field
314 second type of color reference field
316 computing device
318 camera
320 light source
322 processor
324 longitudinal axis
326 tilt angle

1. A computer-implemented method of determining a numerical analyte result value corresponding to a concentration of an analyte in a sample of a bodily fluid of a subject applied to a reagent test region, comprising:
(a) determining a numerical analyte result value based on an image of the reagent test region by using an algorithm which takes into account one or more parameters, each parameter being adapted to take more than one value;
(b) displaying the numerical analyte result value and/or an analyte value range of a group of preset analyte value ranges corresponding to the numerical analyte result value and/or a message corresponding to the corresponding numerical analyte result value or the analyte value range; and
(c) before executing step (a) automatically checking for available updated values for one or more of the parameters on a remote server, and for each available updated value, updating the corresponding parameter value to be taken into account in step (a).

2. The method of claim 1 wherein checking for available update values comprises:
checking on the remote server for each parameter a flag associated with the parameter; or
checking on the remote server at least one status change flag; or
checking on the remote server a status register/flag register; or
comparing each parameter with a corresponding parameter on the remote server.

3. The method of claim 1 wherein checking for available update values comprises: automatically identifying an identification code of a color reference device in the image of the reagent test region and checking for available updates of one or more parameters linked to the identification code.

4. The method of claim 1 wherein at least one parameter is a reference color value of a color field of a color reference device, a failsafe threshold value, or a region of interest margin value.

5. The method of claim 1 wherein when in step (c) a connection to the remote server fails step (a) is executed using the present parameter values and a message indicating the failure to establish a connection to the remote server is displayed.

6. The method of claim 1 wherein the method comprises the step of capturing the image for step (a).

7. A non-transitory computer-readable storage medium including instructions that, when processed by a computing device comprising a processor and a display device, configure the computing device to perform the method of claim 1.

8. The non-transitory computer-readable storage medium of claim 7, wherein step (a) and (c) are performed by the processor and step (b) is performed by the display device.

9. The non-transitory computer-readable storage medium of claim 7, wherein the image of the reagent test region is recorded by a camera comprised by the computing device.

10. A computing device comprising:
a processor, a display device and a communication interface; and
a memory storing instructions that, when executed by the processor, configure the computing device to:
(a) determine, with the processor, a numerical analyte result value based on an image of the reagent test region by using an algorithm which takes into account one or more parameters, each parameter being adapted to take more than one value;
(b) display, by the display device, the numerical analyte result value and/or an analyte value range of a group of preset analyte value ranges corresponding to the numerical analyte result value and/or a message corresponding to the corresponding numerical analyte result value or the analyte value range on the display device; and
(c) before executing step (a), automatically check, by the processor, for available updated values for one or more of the parameters on a remote server, and for each available updated value, updating the corresponding parameter value to be taken into account in step (a).

11. The computing device of claim 10, wherein the computing device comprises a camera adapted to record the image of the reagent test region.
